(19) United States
(12) Patent Application Publication
(10) Pub. No.: US 2010/0125242 A1
(43) Pub. Date: May 20, 2010

(54) EMERGENCY DOSE MEDICATION ADMINISTRATION DEVICE

(75) Inventor: Howard PHYKITT, Rocky Mount, NC (US)

Correspondence Address: INTELLECTUAL PROPERTY / TECHNOLOGY LAW, PO BOX 14329, RESEARCH TRIANGLE PARK, NC, 27709, US

(73) Assignee: PharmStar Pharmaceuticals, Inc., Rocky Mount, NC (US)

(21) Appl. No.: 12/271,874

(22) Filed: Nov. 15, 2008

(51) Int. Cl. A61M 5/31

(52) U.S. Cl. 604/84; 604/88; 604/86

(57) ABSTRACT

An emergency dose medication administration device suitable for administration of an effervescent therapeutic composition, such as a soluble aspirin product that when mixed with water or other aqueous medium forms an effervescent composition for administration to a patient. The administration device of the invention includes separate compartments separated from one another by a barrier member, in which at least one of the compartments includes a bellowed portion which is expansible upon intermixing of materials from the respective compartments of the device, when the barrier member is pierced by a manually actuatable piercing element that includes a passage for administration of the effervescent reaction product to a patient.

### FIELD OF THE INVENTION

The present invention relates to an emergency dose medication administration device, which is useful for administration of emergency medications such as liquid effervescent aspirin compositions in the event of heart attack.

### DESCRIPTION OF THE RELATED ART

In the field of emergency medicine, various therapeutic agents are used, which require immediate administration after a critical physiological event such as heart attack.

Administration of aspirin immediately after onset of cardiovascular symptoms of a heart attack has been credited with saving the lives of numerous heart attack victims. In such circumstance, achieving rapid bioavailability of administered aspirin is of critical importance. For such reason, various powdered aspirin and soluble aspirin products have been developed.

Powdered aspirin products are rapidly taken up by the patient when orally administered, e.g., sub-lingually. Powdered aspirin, however, presents issues of handling and administration, e.g., under windy conditions or when the mouth of the patient is dry and salivary action is insufficient to solubilize the powdered aspirin in the oral cavity, for absorption and uptake through oral cavity mucosa.

Various soluble aspirin products have been developed, such as the stabilized, essentially sodium-free aspirin composition of my U.S. Pat. No. 5,723,453 issued Mar. 3, 1998. Such patent describes an effervescent aspirin composition comprising (i) aspirin granules of a predetermined particle size at a concentration of about 325.0 milligrams to about 1000.0 milligrams per unit dose, and (ii) granular potassium bicarbonate with an outer surface layer of potassium carbonate on the granules thereof, having a concentration of from 250.0 milligrams to about 3000.0 milligrams per unit dose, pH in a range of about 8.0 to about 10.0, and a molar amount in the composition that is greater than a molar amount required to neutralize the aspirin granules.

Although the soluble aspirin product of such prior U.S. Pat. No. 5,723,453 has proven efficacious for use when the powdered composition is added to water or other aqueous medium, it requires opening of a powder package, and addition of water, followed by 30 seconds of dissolution action to yield the drinkable dose of the aspirin solution. This involves a delay in administration time, particularly if a ready source of drinkable water is not available.

It would therefore be a substantial advance in the art to provide an administration device that is simple and effective to administer a bolus of a soluble aspirin composition, without the necessity of locating and using an external source of water.

### SUMMARY OF THE INVENTION

The present invention relates to an emergency dose medication administration device, which is useful for administration of emergency medications such as liquid effervescent aspirin compositions in the event of heart attack.

In one aspect, the invention relates to an emergency dose medication administration device, comprising separate compartments separated from one another by a barrier member, in which at least one of the compartments includes a bellowed portion which is expansible upon intermixing of compositions from the respective compartments of the device to form a therapeutic product, when the barrier member is pierced by a manually actuatable piercing element including a passage for administration of the therapeutic product to a patient.

In another aspect, the invention relates to an emergency dose medication administration device, comprising first and second compartments separated by a barrier member and coupled by an expansible member, with a lower one of the first and second compartments including a distal expansible portion, and with a drinking tube arranged with a piercing member at a distal end thereof for manually-actuated piercing of the barrier member to effect communication between the first and second compartments and intermixing of their contents, to produce a therapeutically effective composition for administration.

In a further aspect, the invention relates to an emergency dose medication administration device, comprising first and second compartments separated by a barrier member and coupled to one another, and a dispensing tube arranged to be manually actuated to disable the barrier member when translated into a dispensing position from a non-actuating position in which the barrier member is enabled to prevent contents of the first and second compartments from intermixing.

Other aspects, features and embodiments of the invention will be more fully apparent from the ensuing disclosure and appended claims.

### BRIEF DESCRIPTION OF THE DRAWINGS

FIG. 1 is a schematic elevation view of an emergency dose medication administration device, according to one embodiment of the present invention.

### DETAILED DESCRIPTION OF THE INVENTION, AND PREFERRED EMBODIMENTS THEREOF

The present invention relates to an emergency dose medication administration device that is usefully employed with therapeutic agents furnished in a two-part formulation, which when mixed with one another generate gas as an effervescent therapeutic composition.

The administration device of the invention in one embodiment includes separate compartments separated from one another by a barrier member, in which at least one of the compartments includes a bellowed portion which is expansible upon intermixing of compositions from the respective compartments of the device, when the barrier member is pierced by a manually actuatable piercing element that includes a passage for administration of the effervescent reaction product to a patient.

The device in one preferred configuration includes first and second compartments separated by a barrier element and coupled by an expansible member, with a lower compartment including a distal expansible portion, and with a drinking tube arranged with a piercing member at a distal end thereof for manually-actuated piercing of the barrier member to effect communication between the first and second compartments and intermixing of their contents, to produce a therapeutically effective composition for administration.

The inventive device may have a variety of alternative forms, in which respective compartments are brought into material mixing relationship with one another by a dispensing (drinking) tube that includes disables the barrier member when translated into a dispensing position.

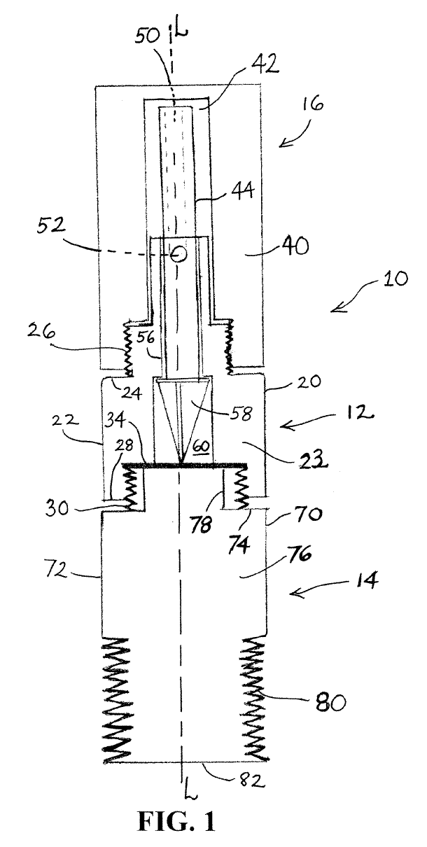

FIG. 1 is a schematic elevational view of an emergency dose medication administration device according to one specific embodiment of the invention.

The device 10 includes a first compartment 12 and a second compartment 14, which are secured to one another by an expansible joint including bellowed member 30 surrounding an upper circumscribing wall 78 of the compartment 14. The bellowed member 30 is joined at one end to an upper neck portion 74 of the cylindrical wall 72 of the housing 70 defining the lower compartment 14. The upper cylindrical wall 78 of compartment 14 is bonded to or integrally formed with the neck 74 of compartment 14.

Attached to cylindrical wall 72 at a lower extremity thereof is a bellowed portion 80, which at its lower end is closed by end wall 82. The walls 72, 78 and 82 together with neck 74 together define the housing 70 of compartment 14.

Overlying and coaxial (along axis L-L) with compartment 14 is compartment 12, including housing 20 comprising cylindrical wall 22 and annular wall 28. Annular wall 28 is joined at its radially inner extremity to bellowed portion 30 interconnecting compartments 12 and 14. Housing 20 also includes an annular neck 24 joined to an upper extremity of cylindrical side wall 22. The annular neck 24 is in turn joined to an exteriorly threaded wall 26 that is threadably engagable with a cap 16 of the device.

The cap 16 includes a main cap body 40 having a cavity 42 therein overfitting drinking tube 44 of the device. In such manner, the cap 16 is readily threadably disengagable from the upper compartment 12 to expose the drinking tube 44 for use.

The drinking tube 44 is provided with a central bore passage 50 communicating with inlet opening 52 in the sidewall of the drinking tube. The drinking tube has a lower portion 56 including a distal spike element 58 reposed in cavity 60.

When the cap 16 is removed from the threaded exterior surface 26 of compartment 12, the upper end of drinking tube 44 is exposed for actuation. Actuation is manually effected by pressing on the upper, proximal end of the drinking tube so that the spike element 58 at the distal end thereof in cavity 60 is driven downwardly, to pierce the barrier element 34 and expose the contents in interior volume 76 of compartment 14 to the contents in the interior volume 23 of compartment 12, so that the materials in the respective compartments intermix with one another.

The intermixing of such materials involves generation of gas that in turn expands the bellows member 80 of compartment 14 and the bellows member 30 between compartments 12 and 14. In this manner, the gas generation is accommodated, and the inlet opening 52 of the drinking tube 44 is disposed in the effervescing liquid, so that liquid passes from the inter-communicated compartments 12 and 14 through inlet 52 into bore passage 50 of drinking tube 44, so that it is available for administration to a patient in need thereof.

In administration, the effervescing liquid generates gas that expands bellows members 30 and 80 so that the device becomes elongated in axial extent (along central line L-L).

The gas-generating action of the effervescing liquid therefore effects gas-driven expulsion of liquid through the bore-passage 50 of drinking tube 44. Such action may be augmented by manual squeezing of the device to longitudinally contract it so that liquid is forced through inlet opening 52 and bore passage 50 in drinking tube 44 into the mouth of a patient. By such action, administration of a bolus of liquid from the device is simply and effectively carried out.

The compartments 12 and 14 may include walls and neck portions that are fabricated from plastic, cellulosic material, metallized plastic, or other suitable material of construction. The bellowed portions 30 and 80 may be formed of a same or different material in relation to the walls and necks of the housing of such compartments. The bellowed portions 30 and 80 may be accordion-folded or in other manner compacted so as to be expansible under mixing and gas generation conditions.

The barrier member 34 may be of any suitable material that is pierceable by the distal piercing element 58 reposed in cavity 60 of the upper compartment 12. For example, the barrier member may be formed of a paper, foil, laminate or polymeric material, as a membrane or closure element that is pierced by the piercing element 58 when downward pressure is applied to the upper end of the drinking tube 44, for movement thereof downwardly along central axis L-L.

The drinking tube 44 is shown as a cylindrical tube having a cylindrical central bore passage 50 therein. It will be recognized, however that the drinking tube may be of various suitable shapes and conformations, including square or other cross-sectional shape and that the proximal end of such tube may be equipped with a flared end, or other conformation rendering it suitable for introduction to the lips or mouth cavity of a subject to whom the effervescent liquid is being administered.

The drinking tube 44 can for example be formed of an injection molded plastic, or alternatively of metal, ceramic or any other suitable material that is compatible with the contents of the respective compartments 12 and 14, and the mixed product of such contents.

The materials in the respective compartments 12 and 14 may be of any suitable type that when intermixed with one another form a therapeutically effective flowable composition.

In application to therapeutic compositions comprising effervescent liquid mixtures, the administration device of the invention is readily manually manipulated to introduce a bolus of the therapeutic composition into the oral cavity of a patient.

While intended primarily for administration to human patients, administration devices of the invention may be usefully adapted for administration of medication to other mammalian and non-mammalian animals, thereby encompassing veterinary, as well as human use, applications.

Any suitable therapeutic agent may be employed. In one embodiment, a first one of the respective compartments contains a powered and/or granular soluble aspirin composition, and the other compartment contains water or other aqueous medium with which the powered and/or granular soluble aspirin composition is reactive to yield an effervescent analgesic liquid product for administration.

It will be recognized that the specific form and configuration of the administration device of the invention may be widely varied, as regards the conformation and arrangement of components, constituents and structural aspects thereof.

While the invention has been has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

What is claimed is:

1. An emergency dose medication administration device, comprising separate compartments separated from one another by a barrier member, in which at least one of the compartments includes a bellowed portion which is expansible upon intermixing of compositions from the respective compartments of the device to form a therapeutic product, when the barrier member is pierced by a manually actuatable piercing element including a passage for administration of the therapeutic product to a patient.

2. The device of claim 1, wherein the compartments are interconnected to one another by a bellowed connection that is expansible upon piercing of the barrier member and intermixing of contents of the separate compartments.

3. The device of claim 1, wherein the barrier member comprises a membrane.

4. The device of claim 1, wherein the compartments are coaxial with one another.

5. The device of claim 1, further comprising a cap that is engageable with one of the compartments to overlie the piercing element.

6. The device of claim 1, wherein the piercing element comprises a hollow bore dispensing passage for administration of the therapeutic product.

7. The device of claim 1, wherein the piercing element includes an opening that communicates with the hollow bore passage, arranged so that when the piercing element has pierced the barrier member, the opening is in flow communication with the compartments, for dispensing of the therapeutic product.

8. The device of claim 1, wherein the piercing element is arranged for downward translation to pierce the barrier member.

9. The device of claim 1, wherein a first one of the compartments contains a dry solids water-soluble aspirin composition, and a second one of the compartments contains an aqueous medium, which in mixture with one another form the therapeutic product as an effervescing analgesic liquid.

10. An emergency dose medication administration device, comprising first and second compartments separated by a barrier member and coupled by an expansible member, with a lower one of the first and second compartments including a distal expansible portion, and with a drinking tube arranged with a piercing member at a distal end thereof for manually-actuated piercing of the barrier member to effect communication between the first and second compartments and intermixing of their contents, to produce a therapeutically effective composition for administration.

11. The device of claim 10, wherein the first compartment contains a dry solids water-soluble aspirin composition, and the second compartment contains an aqueous medium, which in mixture with one another form the therapeutically effective composition as an effervescing analgesic liquid.

12. An emergency dose medication administration device, comprising first and second compartments separated by a barrier member and coupled to one another, and a dispensing tube arranged to be manually actuated to disable the barrier member when translated into a dispensing position from a non-actuating position in which the barrier member is enabled to prevent contents of the first and second compartments from intermixing.
